(19) United States
(12) Patent Application Publication
(10) Pub. No.: US 2010/0180900 A1
(43) Pub. Date: Jul. 22, 2010

(54) SECUREMENT SYSTEM FOR AN ENDOTRACHEAL TUBE

(75) Inventors: Silke Talsma, Atlanta, GA (US); Anthony Coston, Lawrenceville, GA (US); Paul Ciccone, Social Circle, GA (US)

Correspondence Address: KNOBBE MARTENS OLSON & BEAR LLP, 2040 MAIN STREET, FOURTEENTH FLOOR, IRVINE, CA, 92614, US

(73) Assignee: VENETEC INTERNATIONAL, INC., Covington, GA (US)

(21) Appl. No.: 12/664,381

(22) PCT Filed: Jun. 26, 2008
(86) PCT No.: PCT/US2008/068420

§ 371 Date: Dec. 11, 2009

Related U.S. Application Data
(60) Provisional application No. 60/946,173, filed on Jun. 26, 2007.

(51) Int. Cl. A61M 16/00

(52) U.S. Cl. 128/207.14

(57) ABSTRACT

A securement system secures an endotracheal tube or other medical article in position upon a patient and arrests movement of the endotracheal tube. The securement system includes a retainer having a posterior facing surface, and a channel to secure the endotracheal tube. The securement system can include a member configured for placement in the patient's oral cavity and in fluid communication with an anterior side of the retainer, and a mouth securement member configured to substantially prevent movement of the retainer. Advantageously, the securement system can maintain an endotracheal tube in position upon a patient and allow a liquid to be infused into the patient's oral cavity.

### RELATED APPLICATIONS

This application claims the benefit of U.S. Provisional Patent Application No. 60/946,173, filed on Jun. 26, 2007, entitled “ENDOTRACHEAL SECUREMENT DEVICE WITH FLUID RELEASE SYSTEM,” which is hereby incorporated by reference in its entirety.

### BACKGROUND

1. Field of the Invention

The present invention relates generally to techniques and systems for securing an endotracheal tube or other medical line to a patient, and to techniques and systems for infusing a liquid into the oral cavity of the patient when the medical line is secured to the patient.

2. Description of the Related Art

Endotracheal tubes are commonly administered to medical patients, for example to mechanically-ventilated patients. Placement of an endotracheal tube into the throat of a patient is referred to as intubation. Failing to secure the endotracheal tube properly, however, can result in the migration of the tube within the throat of the patient. Such motion is undesirable since outward motion of the tube can result in the tube moving entirely out of the airway of the patient, eliminating its effectiveness and potentially ventilating the patient's gastro-intestinal tract instead of his lungs. Inward motion of the tube is undesirable because it will eventually result in the tube moving down one of the patient's bronchi, preventing air from being ventilated to the other lung. This will quickly lead to the collapse of the unventilated lung. Even slight back and forth motion of the tube within the throat can result in tracheomalacia and ultimately in the weakening or collapse of the trachea, which may require permanent stenting to maintain an open airway. Migration may further result in a form of nosocomial pneumonia.

Nosocomial pneumonia, also referred to as hospital-acquired pneumonia, is a serious complication in mechanically-ventilated patients. Pneumonia is one of the most common nosocomial infections in the United States, and results in substantial morbidity and mortality. The risk for pneumonia is increased by the direct access of bacteria to the lower respiratory tract. This occurs because of leakage around the cuff of an administered endotracheal tube, thereby enabling pooled secretions to enter the trachea. Pneumonia is frequently caused by the aspiration of mouth secretions into the upper airways.

In order to avoid these undesirable consequences, it is common to secure the endotracheal tube in position upon the patient by means of adhesive tape. For instance, a medical practitioner may, after positioning the tube within the throat of the patient, wrap adhesive tape around the tube and tape it to the patient. Various devices are often used as adaptors for endotracheal tubes, and sometimes these devices are taped to the patient instead of directly taping the endotracheal tube to the patient.

Such arrangements present certain problems for a medical practitioner. One difficulty is that once secured, it is generally not convenient to release or adjust the securement of the endotracheal tube. For instance, if an adhesive tape is used to secure the tube to the patient, adjusting or re-securing the tube in such situations requires that the adhesive tape be removed from the patient and the tube, and then fresh adhesive tape be used to properly re-secure the tube upon the patient. Such adhesive tape can be difficult to work with for medical practitioners wearing latex gloves, and contact with the adhesive can introduce tears or microscopic perforations into the gloves, compromising their effectiveness.

Furthermore, the use of adhesive tape may be harmful to the patient. Repeated application and removal of adhesive from the face of a patient can irritate or damage the skin of the patient, which is a particular risk for long-term ventilated patients. In addition, adhesive tape can harbor a significant number of bacteria around a patient's skin. Furthermore, adhesive tape may not effectively prevent repositioning and unplanned extubation of the endotracheal tube. As discussed above, migration of the tube within the throat of the patient is undesirable, and unplanned extubation or repositioning of the tube can cause secretions to leak around an inflation cuff of the tube and thereby leak into a patient's upper airways.

After an endotracheal tube is attached to a patient, access to the patient's mouth may be impaired by the endotracheal tube. However, allowing a medical practitioner access to the patient's mouth, for example to provide oral care, is desirable because ventilated patients are typically unable to care for themselves, and thus are unlikely to exercise proper oral care.

Improper oral care can facilitate the colonization of harmful bacteria in the oral cavity. The mouth is colonized with hundreds of microorganism species. The endotracheal tube provides a surface on which these microorganisms can colonize and establish a biofilm. While concentrations of microorganisms in the oral cavity are generally regulated by competitive inhibition among species, the introduction of a foreign surface such as an endotracheal tube may alter the balance of microorganisms in the oral cavity. Furthermore, the endotracheal tube may provide a pathway for microorganisms to migrate. Even microorganisms that normally colonize in the oral cavity may cause harm to the patient if allowed to migrate, such as to the lower airways.

The problem of bacteria colonization may be exacerbated in an intubated patient because normal immune defense mechanisms are impaired in such patients. For example, saliva production, the ability to swallow, and the cough reflex may all be impaired. Such impairment allows microorganisms increased access to the lower airways.

Medical practitioners, such as nurses or respiratory technicians, are expected to clean a patient's oral cavity at regular intervals, e.g., every four hours. Oral care of an intubated patient, however, is a difficult and time-consuming task for medical practitioners, many of whom are consumed with other high-priority tasks.

Therefore, a need continues to exist for an improved system to secure an endotracheal tube to a medical patient and for a system that allows for improved oral care of a patient.

### SUMMARY

One aspect of the present invention thus involves a retainer for an endotracheal tube. The retainer includes a posterior facing surface configured to inhibit movement of the retainer into a person's mouth; a channel configured to receive a portion of the endotracheal tube so as to inhibit movement of the endotracheal tube relative to the retainer; a distribution manifold, at least a portion of the distribution manifold being configured to be disposed in the person's mouth; and a lumen, at least a portion of the lumen being disposed in the retainer so that a liquid passing through the lumen enters the person's mouth.

Another aspect involves an endotracheal tube securement system. The securement system includes a head contact member configured to be secured to a patient and a retainer securable to the head contact member. The retainer includes a support member having a posterior facing surface configured to inhibit movement of the retainer relative to a patient's mouth, an aligning member configured to receive a portion of the endotracheal tube so as to inhibit movement of the endotracheal tube in at least one direction relative to the support member, and a lumen disposed so that a liquid passing through the lumen enters the person's mouth.

Yet another aspect involves a retainer for securing an endotracheal tube to a patient. The retainer includes an alignment member configured to inhibit movement of the endotracheal tube relative to the retainer, a first aperture configured to be disposed within a patient's mouth, and a second aperture configured to be accessible from outside the patient's mouth and at least partially in flow communication with the first aperture.

Further aspects, features and advantages of the present invention will become apparent from the detailed description of certain embodiments that follows.

### BRIEF DESCRIPTION OF THE DRAWINGS

The above mentioned and other features of the invention will now be described with reference to the drawings of several embodiments of the present securement system. The illustrated embodiments of the securement system are intended to illustrate, but not to limit the invention. The drawings contain the following figures:

FIG. 1 is a perspective view of a securement system in accordance with a preferred embodiment of the present invention and shows a retainer, a porous structure, a mouth securement member, and flexible bands.

FIG. 2 is a perspective view of the securement system from FIG. 1 with the porous structure, the mouth securement member, and the flexible bands removed.

FIG. 3a is a front view of the retainer of FIG. 2 and shows a hinged member of the retainer engaged with a stationary member of the retainer.

FIG. 3b is another front view of the retainer of FIG. 2 and shows the hinged member from FIG. 3a disengaged from the stationary member.

FIG. 4 is a back view of the retainer from FIG. 2.

FIG. 5 is a top view of the retainer from FIG. 2 and shows a lumen extending through the retainer and outlined in dashed lines.

FIG. 6 is a bottom view of the retainer from FIG. 2.

FIG. 7 is a side view of the retainer from FIG. 2.

FIG. 8 is a cross-sectional view of the retainer of FIG. 2, taken along line 8-8 of FIG. 5 to expose the lumen in the retainer.

FIG. 9 is a perspective view of an upper portion of the porous structure from FIG. 1.

FIG. 10 is a perspective view of a lower portion of the porous structure from FIG. 1.

FIG. 11 is a perspective view of the mouth securement member from FIG. 1.

FIG. 12 is a top view of the mouth securement member of FIG. 11.

FIG. 13 is a bottom view of the mouth securement member of FIG. 11.

FIG. 14 is a front view of the mouth securement member of FIG. 11.

FIG. 15 is a back view of the mouth securement member of FIG. 11.

FIG. 16 is a side view of the mouth securement member of FIG. 11.

FIG. 17 is a perspective exploded view of the securement system of FIG. 1 with the flexible bands removed.

FIG. 18a is a perspective assembled view of the securement system of from FIG. 17.

FIG. 18b is a cross-sectional view through the retainer of FIG. 18a along line 18b-18b.

FIG. 19 is a perspective view of the flexible band and adjustment member from FIG. 1.

FIG. 20 is a top view of the flexible band of FIG. 19.

FIG. 21 is a perspective view of the adjustment member from FIG. 19.

FIG. 22 is a perspective view of the securement system of FIG. 1 attached to a patient.

FIG. 23 is a perspective view of another embodiment of the securement system and shows a retainer, a mouth securement member, and a strap.

FIG. 24 is a perspective view of the retainer from FIG. 23.

FIG. 25 is a top view of the retainer from FIG. 24.

FIG. 26 is a bottom view of the retainer from FIG. 24.

FIG. 27 is a front view of the retainer from FIG. 24.

FIG. 28 is a back view of the retainer from FIG. 24.

FIG. 29 is a cross-sectional view of the retainer of FIG. 24, taken along line 29-29 of FIG. 25.

FIG. 30 is a top view of the strap of FIG. 23.

FIG. 31 is a bottom view of the strap of FIG. 30.

FIG. 32 is a side view of the strap of FIG. 30.

FIG. 33 is a perspective view of the securement system of FIG. 23 attached to a patient.

FIG. 34 is a perspective view of another embodiment of the securement system of FIG. 23 attached to a patient with a plurality of straps.

### DETAILED DESCRIPTION OF CERTAIN EMBODIMENTS

The following description and examples illustrate preferred embodiments of the present securement system disclosed in the context of use with an exemplary endotracheal tube. More specifically, the embodiments relate to a securement system and related techniques that maintain an endotracheal tube in position upon a patient and/or that inhibit migration of the tube within the throat of the patient. The securement system is configured to allow a liquid to be infused into the patient's oral cavity. The principles of the present invention, however, are not limited to endotracheal tubes such as those shown. It will be understood by those of skill in the art in view of the present disclosure that the securement system described can be used with other types of medical articles, including, but not limited to endotracheal tubes of different design, either with or without tube adaptors, and the like. One skilled in the art may also find additional applications for the devices and systems disclosed herein. Thus, the illustration and description of the securement system in connection with an endotracheal tube is merely exemplary of one possible application of the securement system and technique disclosed.

The securement system described herein not only secures an endotracheal tube to a medical patient, it may also inhibit occlusion of the endotracheal tube. The endotracheal tube is deformable and passes between the patient's teeth; thus, there is a risk that the tube may become occluded by a patient's teeth when the patient attempts to bite down. Such occlusion can lead to, for example, hypoxia. The present securement system may be configured to inhibit this occlusion using a blocker to prevent the patient from biting down on the endotracheal tube.

Furthermore, the securement system described herein allows for improved oral care of a patient. To facilitate improved oral care, the system permits the infusion of a liquid, such as an antimicrobial agent, into the oral cavity. Advantageously, the liquid can be administered without removing the endotracheal tube.

The liquid can be chosen from any number of liquids normally infused in an oral cavity. For example, the liquid can be an antimicrobial agent such as chlorhexidine or a sale thereof, such as chlorhexidine gluconate. Using chlorhexidine to prophylactically decolonize the oral cavity of heart surgery patients is recommended by the Centers for Disease Control and Prevention (CDC). The antimicrobial agent can also be hydrogen peroxide. Additional liquids include moisturizing agents and fluoridation agents. The amount of liquid infused into the patient will depend, of course, on the liquid. In the case of chlorhexidine gluconate, the amount may range from 2 cc's to 20 cc's, for example 5-10 cc's every four hours or as otherwise needed.

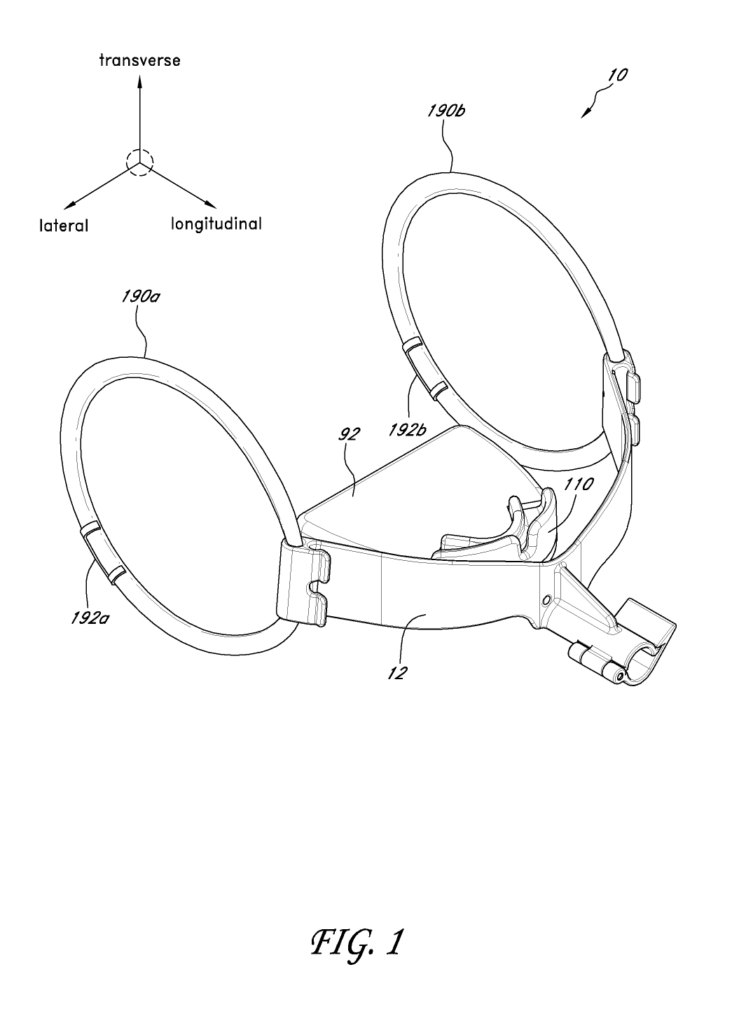

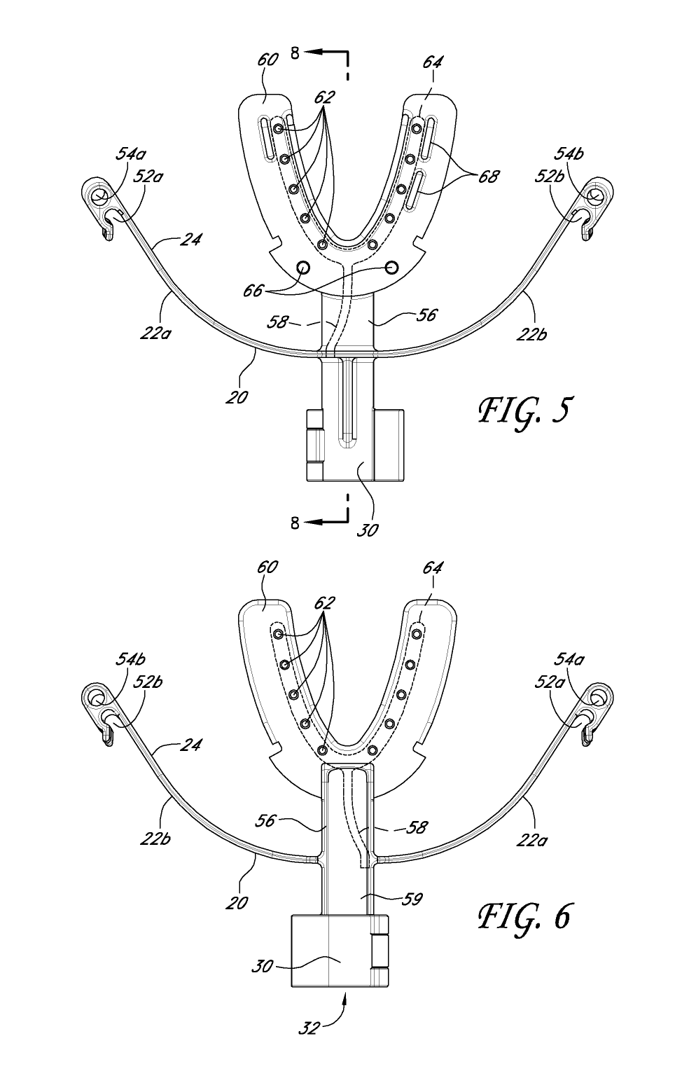

A detailed description of embodiments of a securement system, and its associated method of use, now follows. With reference now to FIG. 1, an embodiment of a securement system 10 includes a retainer 12 that has one or more grooves, channels or lumens 64 as is illustrated in FIG. 5. The one or more grooves, channels or lumens 64 provide one or more passageways through at least a portion of the retainer 12. The one or more passageways connect one or more locations that are outside of the oral cavity of the patient with one or more locations that are inside the oral cavity. In this way, a medical practitioner is able to infuse a liquid into the patient's mouth via one or more passageways when the retainer 12 is positioned in the patient's mouth.

For ease of explanation, the term lumen is used in a generic sense to describe the one or more grooves, channels, passageways, etc. that separately or together form at least a portion of a path into the oral cavity. The lumen 64 need not, but it may, form a closed passageway into the oral cavity. For example, a wall of the lumen 64 may have an opening or slot at one or more locations along the longitudinal axis of the lumen 64. Thus, the lumen 64 may be closed and opened for different portions of the longitudinal length of the lumen 64.

The shape and cross-sectional area of the lumen may vary along the length of the lumen 64. The lumen 64 may transition between multiple shapes along its length. For example, the lumen 64 may transition between a first shape and a second shape that is different than the first shape. A portion of the lumen 64 may have a generally semi-circular cross-sectional shape. Of course, the lumen 64 may have a constant cross-section along its entire length. The lumen 64 may form one or more manifolds or pockets in the retainer 12.

The shape and cross-sectional area of the lumen 64 may be selected depending on, for example, the expected amount of liquid to be administered to the patient, the desired rate of flow through the retainer 12, the viscosity of the liquid, and/or the ambient temperature. Depending on the shape of the lumen 64, the lumen 64 may be capable of facilitating flow of the liquid into the patient's oral cavity. In contrast, in a retainer 12 configured to slowly release liquid into the patient's oral cavity, the lumen 64 may have a tapering inner surface and a radius that narrows in a direction towards the oral cavity.

Additional embodiments of the lumen 64 of the retainer 12 can comprise a plurality of different radii, sizes, and/or tapering regions. For example, the lumen 64 can have two sections: a first proximal section have a generally uniform cross-sectional size along its length while a second distal section has a tapering shape along its length. A manifold or collection region can form a transition between these two sections of the lumen 64. These sections of the lumen 64 can also both be tapered or straight or the distal section can be straight and the proximal section can be tapered. In this way, the size and shape of the lumen 64 can be chosen to facilitate or inhibit liquid flow through the retainer 12. By selecting the inner surface contour of the lumen 64 in view of the desired flow characteristics and/or viscosity of the liquid, a more effective treatment may be achieved.

The retainer 12 may include a single lumen 64 or multiple lumens. For embodiments of the retainer 12 that have multiple lumens, the lumens may or may not be interconnected. For example, multiple lumens may form one or more branching sites where a single lumen separates into two or more lumens or two or more lumens combine to form fewer lumens. At least a portion of one of the lumens 64 is disposed in the retainer 12.

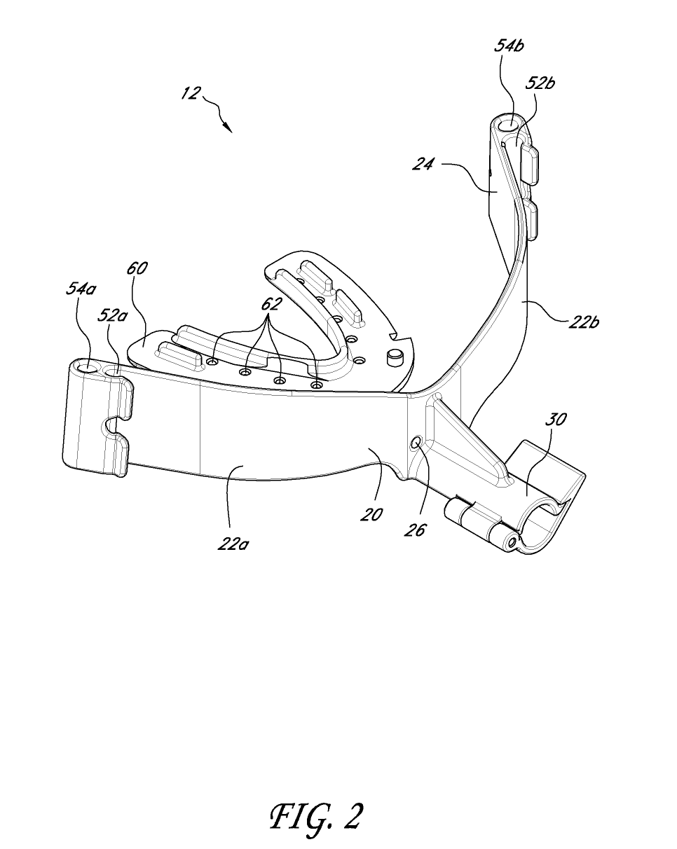

FIG. 2 is a perspective view of the securement system 10 from FIG. 1 with the porous structure 92, the mouth securement member 110, and the flexible bands 190a, 190b removed to more clearly show a distribution manifold 60. In the illustrated embodiment, at least a portion of the lumen 64 extends into the distribution manifold 60. The lumen 64 forms a passageway between an entrance aperture 26 and one or more exit apertures 62. The one or more exit apertures 62 may be disposed, for example, on top and/or bottom surfaces of the distribution manifold 60 or on a proximal side of the mouth securement member 110.

The lumen 64 passes through the retainer 12 between apertures 26, 62. In the illustrated embodiment, a portion of the lumen 64 connecting the apertures 26, 62 passes through connection member 56. Thus, a liquid entering aperture 26 will flow through the connection member 56 and out through one or more apertures 62 into the patient's mouth.

In other embodiments, the lumen 64 does not extend into the distribution manifold 60. For example, the liquid may be directly added to the distribution manifold 60 in other ways, such as from the top of the distribution manifold 60. In such an embodiment, a portion of the distribution manifold 60 may be dispose outside the patient mouth so as to allow access to the distribution manifold 60 without removing the endotracheal tube. For example, the exposed portion of the distribution manifold 60 may have an aperture or opening that connects with the one or more apertures 62.

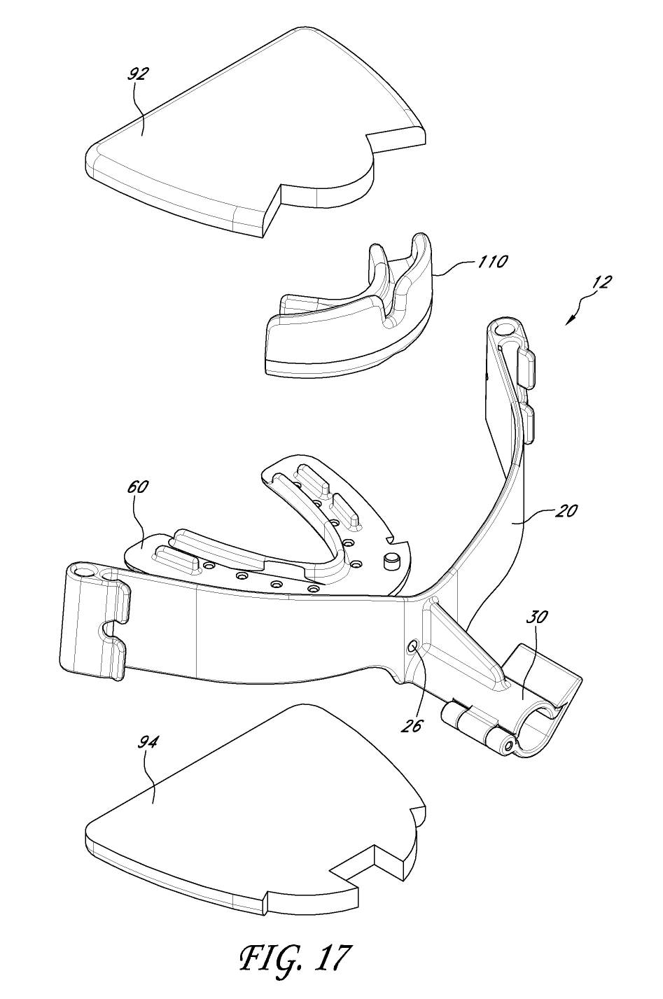

Returning to FIG. 1, the securement system 10 may further include flexible bands 190a and 190b, a porous structure, and a mouth securement member 110. The porous structure may comprise an upper portion 92 and a lower portion 94 as is most clearly shown in FIG. 17. The retainer 12 is configured to be secured to the patient and inhibit further movement of the retainer 12 into the patient's mouth once the retainer 12 is secured.

The flexible bands 190a and 190b are configured to hook over at least a portion of the patient's ears and secure the retainer 12 to the patient's face. The adjustment members 192a and 192b are configured to secure and adjust a circumference of the flexible bands 190a and 190b, respectively. The upper and lower porous structures 92, 94 and the mouth securement member 110 are configured for placement in the patient's mouth.

To assist in the description of the components of embodiments of the securement system, the following coordinate terms are used, consistent with the coordinate axes illustrated in FIG. 1. A “longitudinal axis” is generally parallel to a portion of a medical article retained by the securement system 10. A “lateral axis” is normal to the longitudinal axis and is generally tangential to the curve of the retainer 12 where the portion of the medical article would cross the retainer 12. When the securement system 10 is secured to the patient, the lateral axis will be generally parallel to a width of the patient's face (i.e. from cheek to cheek).

A “transverse axis” extends normal to both the longitudinal and lateral axes. When the securement system 10 is secured to the patient, the transverse axis will be generally parallel to a length of the patient's face (i.e. from chin to forehead). In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. Also, the terms “proximal” and “distal”, which are used to describe the present securement system 10, are used consistently with the description of the exemplary application. Thus, proximal and distal are used in reference to the center of the patient's body.

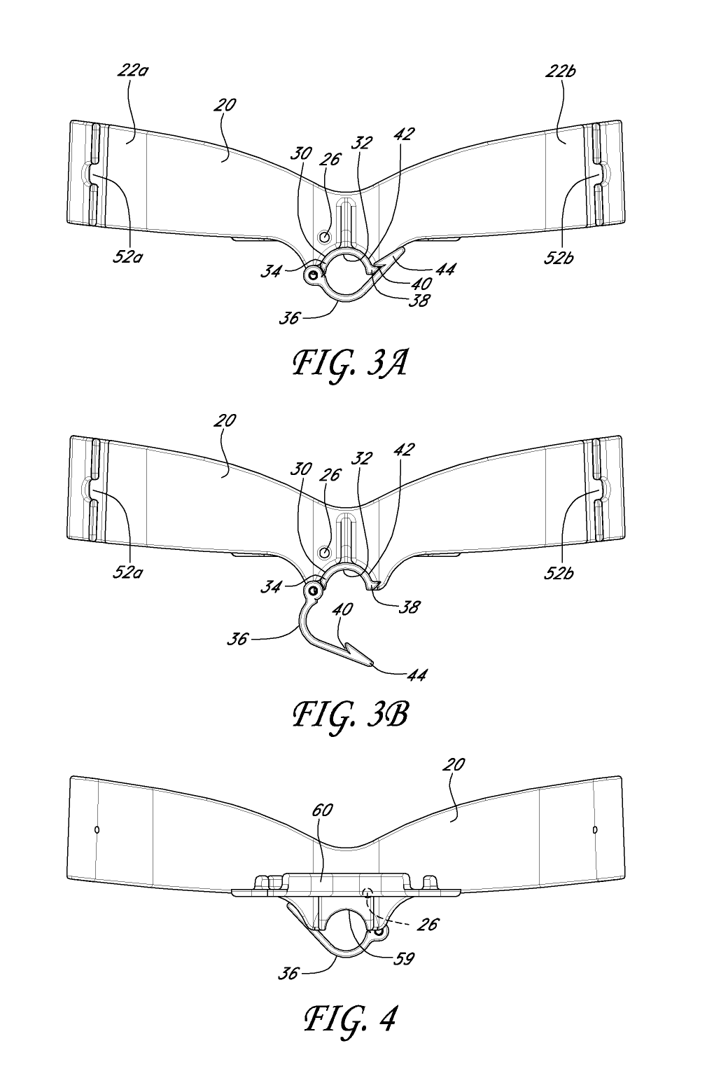

FIGS. 3-8 further illustrate the retainer 12 from FIG. 2. The retainer 12 includes a support member 20, an aligning member 30, and a distribution manifold 60. The support member 20 has a posterior facing surface 24 configured to inhibit movement of the retainer 12 into the patient's mouth. The aligning member 30 is configured to receive a portion of an endotracheal tube such that the tube is aligned with the patient's mouth. The distribution manifold 60 is configured to permit infusion of liquid into the patient's mouth.

The support member 20 is comprised of a first side 22a and a second side 22b, which together form a generally curved shape. Configuring the support member 20 as a generally curved shape enhances the fit of the support member 20 with the patient's face. Of course the support member 20 is not limited to a curved shape and may have any other shape.

In the illustrated embodiment, the first side 22a and second side 22b are substantially symmetrical. The first side 22a and the second side 22b may also be asymmetrical. Such a design may be advantageous, for example, to accommodate different facial shapes or to accommodate medical equipment that may be positioned on a side of the face. Those skilled in the art will appreciate that the first side 22a and the second side 22b may each be shorter or longer, or the proximal ends of the first side 22a and the second side 22b may be spaced closer together or further apart. Such differing configurations of the support member 20 may accommodate differing facial shapes or differing medical applications, for example.

The support member 20 is configured to secure to a plurality of straps or bands of a head contact member. The head contact member need not wrap around the entire head. For example, the head contact member may wrap around the ears of the patient.

In the illustrated embodiment, the ends of the support member 20 accept a portion of a flexible band, such as either of the flexible bands 190a or 190b illustrated in FIG. 1. As can be seen in the top and bottom views of the retainer 12 in FIGS. 5 and 6, respectively, each proximal end of the support member 20 may be configured to define a receiving space 52a or 52b to receive a portion of the flexible band 190a, 190b.

The receiving spaces 52a and 52b each comprise an opening defined by a portion of the support member 20 being curved toward a distal/anterior side of the support member 20. In this configuration, a medical practitioner can place the flexible band against the distal side of the support member, and then pull the flexible band towards the receiving space 52a or 52b to engage the support member 20 with the flexible band.

As also illustrated in FIGS. 5 and 6, each proximal end of the support member 20 may be configured to define a receiving space 54a or 54b to receive a portion of the flexible band 190a, 190b. The receiving spaces 54a and 54b each comprise a hole formed through the support member 20. In this configuration, a medical practitioner can feed an end of the flexible band through the receiving space 54a or 54b before forming the flexible band into a loop. Alternatively, the support member 20 may be manufactured with the flexible bands 190a, 190b so that the medical practitioner receives the retainer 12 with the flexible bands already attached to the retainer 12 via the receiving spaces 54a, 54b. Such configuration diminishes the chances of the flexible band accidentally disconnecting from the support member 20. Those of skill in the art will understand that the proximal ends of the support member 20 may be configured to define only receiving spaces 52a and 52b, only receiving spaces 54a and 54b, or a combination of the two types of receiving spaces.

The support member 20 is configured to define an opening or aperture 26. The aperture 26 provides access to the lumen 64. The lumen 64 provides a passageway to the patients oral cavity from a distal side of the support member 20, thereby allowing infusion of a liquid into the patient's oral cavity. In certain embodiment, the aperture 26 is disposed in the mouth securement member 110. Access to the aperture 26 in the mouth securement member 110 is provided through or around the support member 20. For example, the width of the securement member 20 may be reduced in the region of the mouth securement member 110 so as to provide unobstructed access to the aperture 26 in the mouth securement member 110. Alternatively, an opening may be provided in the support member 20 that is positioned over the aperture 26 in the mouth securement member 110.

A medical practitioner may eject a liquid, such as an antimicrobial agent, into the aperture 26 in order to flush the patient's oral cavity with the liquid. The liquid may be ejected in to the aperture 26 by use of a syringe, a bottle used to dispense liquid, or by any other means configured to dispense liquid into an aperture.

The aligning member 30 is attached to the support member 20. As can be seen in FIG. 3a, the aligning member 30 forms at least a partial channel 32 that receives at least a portion of an endotracheal tube. The aligning member 30 substantially inhibits the endotracheal tube from moving in at least a longitudinal direction when the endotracheal tube is placed in the aligning member 30 and the aligning member is closed around the endotracheal tube. The aligning member 30 preferably further inhibits movement of the endotracheal tube in lateral and transverse directions.

The aligning member 30 releasably secures the endotracheal tube. A stationary member 34 and a movable member 36 are configured to form the channel 32 when in a closed position. When in the closed position, a protrusion 38 engages with an indentation 40 in hinged member 36. Those of skill in the art will understand that a protrusion on the hinged member 36 may instead engage with an indentation in the stationary member 34, that both the stationary member 34 and the hinged member 36 may comprise protrusions, or that other engagement means may be selected. In other embodiments, the hinged member 36 of the aligning member 30 may be omitted.

To disengage the hinged member 36 from the stationary member 34, a medical practitioner applies pressure towards the channel 32 in an area 42 of the stationary member 34. Alternatively, the medical practitioner may apply pressure away from the channel 32 to an area 44 of the hinged member 36. After the hinged member 36 disengages from the stationary member 34, the aligning member 30 may appear as illustrated in FIG. 3b in an open position.

When the hinged member 36 is disengaged from the stationary member 34, a medical practitioner may introduce a portion of an endotracheal tube into the channel 32. Thereafter, the medical practitioner may press the hinged member 36 towards the channel 32 and apply sufficient pressure to the hinged member 36 to cause the hinged member 36 to engage the stationary member 34. Thus, the aligning member will enclose the portion of the tube when the hinged member 36 and the stationary member 34 are engaged, thereby surrounding the circumference of the tube.

In the illustrated embodiment, an enclosed area formed when the hinged member 36 and the stationary member 34 engage is roughly circular. In this embodiment, the aligning member 30 can securely surround an endotracheal tube and lock the tube into position when the stationary member 34 and the hinged member 36 are engaged. In other embodiments, the enclosed area may be elliptical, rectangular, or of another shape. Such configurations may be configured to substantially prevent lateral, transverse, and/or longitudinal motion of an endotracheal tube when the tube is enclosed by the aligning member 30.

In the illustrated embodiment, the area 44 of the hinged member 36 is located on a side opposite that of the aperture 26 formed in the support member 20. Such location of the area 44 and the aperture 26 decreases the likelihood of the aperture 26 being obstructed by the hinged member 36, and decreases the likelihood of the hinged member 26 interfering with a medical practitioner who is ejecting a liquid into the aperture 26. In other embodiments, the area 44 and the aperture 26 may be located on the same side of the support member 20.

The distribution manifold 60 is attached to a proximal side of the support member 20 by a connection member 56, as can be seen in the top view of the support member 20. In some embodiment, the connection member 56 not only connects the distribution manifold 60 to the support member 20, but may also form a bite block. The bite block blocks a patient's teeth from contacting the endotracheal tube in the patient's mouth. Thus, if the patient were to bite down, the connection member 56 would diminish the possibility of the tube being punctured or occluded.

In the illustrated embodiment, the connection member 56 includes a channel 59. The channel 59 is preferably coaxially aligned with the channel 32 in the aligning member 30. The channel 59 accepts a portion of the endotracheal tube. The channel 59 can be shaped in any way that allows the endotracheal tube to be placed at least partially therein. In the illustrated embodiment, the channel 59 is generally semicircular, but other shapes may be selected.

Furthermore, the lumen 64 may include a portion 58 disposed in the connection member 56. The portion 58 of the lumen may be configured to direct the liquid, administered by a medical practitioner at a distal/anterior side of the support member 20, such as at the aperture 26, to the distribution manifold 60. As can be seen in a cross-sectional view of the retainer 12, the connection member 56 illustrated in FIG. 8 defines the portion 58 of the lumen 64 which allows passage of the liquid to the distribution manifold 60.

Any portion of the lumen 64, including the portion 58, may include a valve. The valve may be a one-way valve that allows passage of liquid towards the distribution manifold 60, but not back towards the aperture 26. In other embodiments, the valve may be omitted. In yet other embodiments, the connection member 56 may be configured to define a full or partial channel or conduit to allow passage of the liquid to the distribution manifold 60. Alternatively, a channel, lumen, or passageway separate from the connection member 56 may be configured to allow passage of the liquid from the aperture 26 to the distribution manifold 60.

The distribution manifold 60 includes at least one aperture 62. In the illustrated embodiment, the distribution manifold 60 includes a plurality of apertures 62. As can be seen in a top view of the distribution member 60, the apertures 62 are preferably dispersed throughout the distribution manifold 60. The dispersion of holes increases the likelihood that a liquid applied to the distribution manifold 60, such as a liquid directed to the distribution manifold 60 by the connection member 56, will be distributed throughout the patient's oral cavity.

In the illustrated embodiment, the distribution manifold 60 is configured as a generally curved plate. This curved shape roughly mirrors the shape of the inside of a patient's mouth. Thus, the distribution manifold 60 can fit comfortably inside the patient's oral cavity and the distribution of liquid throughout the oral cavity can be improved. A liquid applied to the distribution manifold 60 will travel along the distribution manifold 60 and may exit from the apertures 62, or may exit along the edges of distribution manifold 60. Thus, a medical practitioner may eject liquid into the aperture 26 defined in the support member 20 at a single location or on a single side of the patient's face and achieve a distributed infusion of the liquid within the patient's oral cavity upon the liquid exiting the distribution manifold 60.

In the illustrated embodiment, at least a portion of the lumen 64 passes through the distribution manifold 60. As explained above, the lumen 64 is preferably in fluid communication with the apertures 26 and 62. The connection member 56 may include a portion 58 of the lumen 64. Thus, a liquid entering the aperture 26 will flow through the lumen 64 and out through the aperture 62 into the patient's mouth. In other embodiments, the lumen 64 may be omitted from the distribution manifold 60 and/or liquid may be applied to the distribution manifold 60 in other ways, such as to the top of the distribution manifold 60.

Optionally, the distribution manifold 60 may comprise one or more raised areas or protrusions 66 and 68. The protrusions 68 may be configured to guide the flow path of liquid exiting the apertures 62 across the distribution manifold 60. The protrusions 68 may be employed with or without embodiments of the retainer 12 that do not a portion of the lumen 64 in the distribution manifold 60. Thus, the protrusions 68 may be employed in addition to or in place of the apertures 62 when the lumen 64 is omitted from the distribution manifold 60. For example, the lumen 64 could terminate at the proximal end of the lumen portion 58 and flow across a surface of the distribution manifold 60. The distribution manifold 60 could include one or more grooves or channels in its top surface to guide the flow of liquid toward the proximal side of the distribution manifold 60.

In one embodiment, the distribution manifold 60 may be shaped as a single bar or may comprise a single protrusion to divert the path that liquid travels. In other embodiments, a plurality of protrusions or channels may alter the liquid path.

The protrusions 66 and 68 may aid in attaching other structures to the distribution manifold 60. For example, the protrusions 66 may fit within corresponding receptacles in the mouth securement member 110. Upper and or lower protrusions 68 may fit with corresponding receptacles in the upper or lower portions of the porous structure 92, 94.

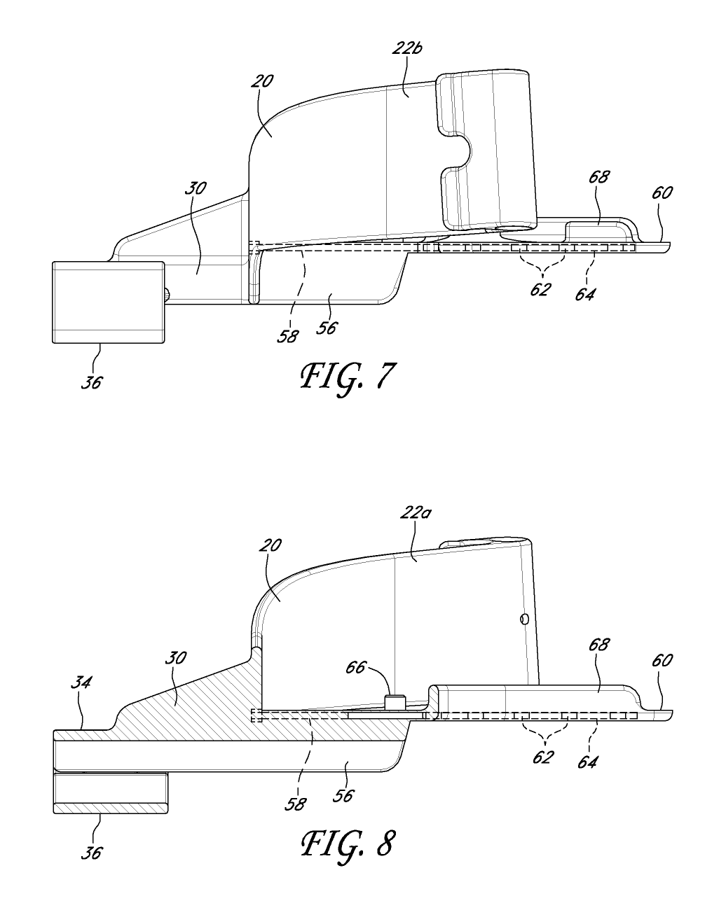

In the illustrated embodiment, the support member 20, the aligning member 30, and the distribution manifold 60 are integrally formed, as can be seen in a side view and a cross-sectional view, illustrated in FIGS. 7 and 8. Such an integrally formed structure may be manufactured by any number of processes known in the art, such as by a molding process for example. In other embodiments, the support member 20, the aligning member 30, the distribution manifold 60, and any components thereof, may be separately formed and attached together. Suitable materials for the members and components thereof may include, for example, but without limitation, plastics, polymers or composites such as polypropylene, polyethylene, polycarbonate, polyvinylchloride, acrylonitrile butadiene styrene, nylon, olefin, acrylic, polyester, as well as moldable silicon, thermoplastic urethane, thermoplastic elastomers, thermoset plastics and the like. However, other materials can be utilized.

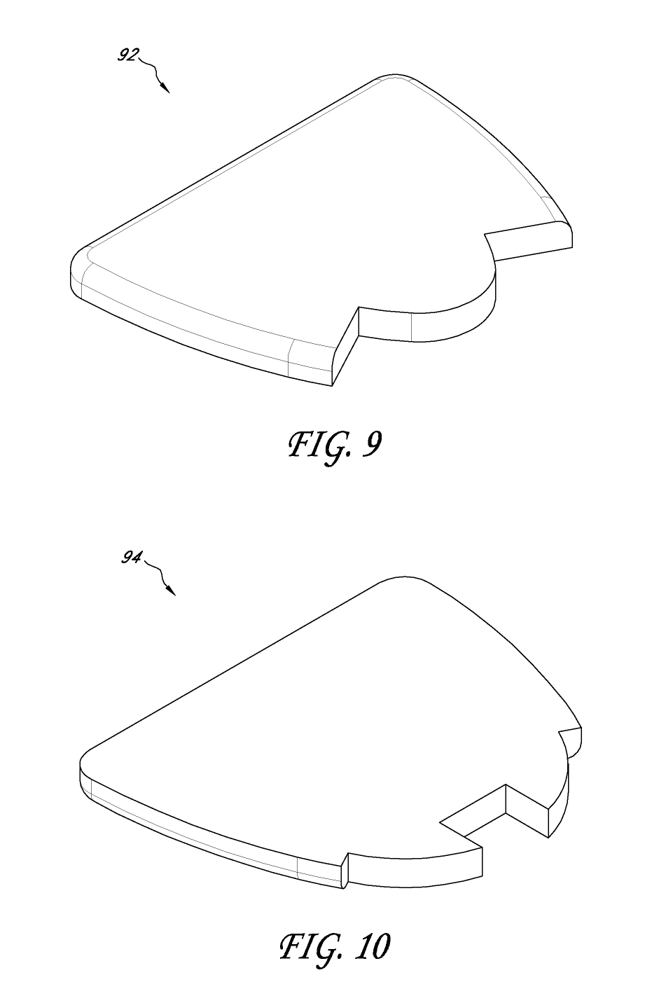

The securement system 10 may comprise an upper portion 92 and a lower portion 94 forming the porous structure. FIG. 9 is a perspective view of the upper portion 92 of the porous structure of FIG. 1. The upper porous structure 92 is configured to attach to an upper surface of the distribution manifold 60. The upper porous structure 92 may interact with the lower porous structure 94 as illustrated in FIG. 10 and/or the mouth securement member 110 as illustrated in FIGS. 1 and 11. In the illustrated embodiment, the upper porous structure 92 is configured to attach to the upper surface of the distribution manifold 60 and will cover the upper apertures 62.

The upper porous structure 92 is configured to communicate liquid from the distribution manifold 60 to the patient's oral cavity. A liquid traveling along the distribution manifold 60 or exiting from the upper apertures 62 will contact the upper porous structure 92, which will communicate the liquid, thereby wetting at least a portion of the patient's oral cavity. In addition to this communication of liquid, the upper porous structure 92 may aid in the distribution of the liquid within the patient's oral cavity or may increase the amount of time necessary for a liquid administered to the distribution manifold 60 to reach the patient's oral cavity. To effect such liquid communication, and increase liquid distribution or time necessary for liquid communication, the upper porous structure 92 may comprise a sponge-like material or other porous material.

FIG. 10 illustrates the lower portion of the porous material 94 which attaches to the retainer 12. The lower porous structure 94 may be configured similar to the upper porous structure 92 and communicate liquid and similarly interact with other structures that attach to the distribution manifold 60. In the illustrated embodiment, the lower porous structure 94 attaches to a lower surface of the distribution manifold 60 and will cover the lower apertures 62. The illustrated embodiments of the porous structure 92 and the porous structure 94 may be simultaneously attached to the top and bottom of the distribution manifold 60, respectively, thereby enclosing the tops and bottoms of the apertures 62.

The securement system 10 also may comprise a mouth securement member 110. FIG. 11 is a perspective view of the mouth securement member 110 illustrated in FIG. 1. The mouth securement member 110 is configured to attach to the retainer 12 for placement in the patient's mouth.

In one embodiment, the mouth securement member 110 is configured to receive a patient's teeth, at least one of the patient's gums, or both. When a patient's teeth and/or gums are received by the mouth securement member 110, the mouth securement member 110 may further prevent substantial movement of the securement system 10 relative to the patient in addition to the securement provided by the securement member 20. The mouth securement member 110 may include structure that blocks a patient's teeth from contacting an endotracheal tube in the patient's mouth. As can be seen in a back view of the mouth securement member 110, illustrated in FIG. 15, the front of the mouth securement member 110 may be higher than the back, which improves the likelihood that the front will contact a patient's teeth and/or gums when placed in the patient's mouth and thereby further decreases the likelihood of movement of the securement system 10.

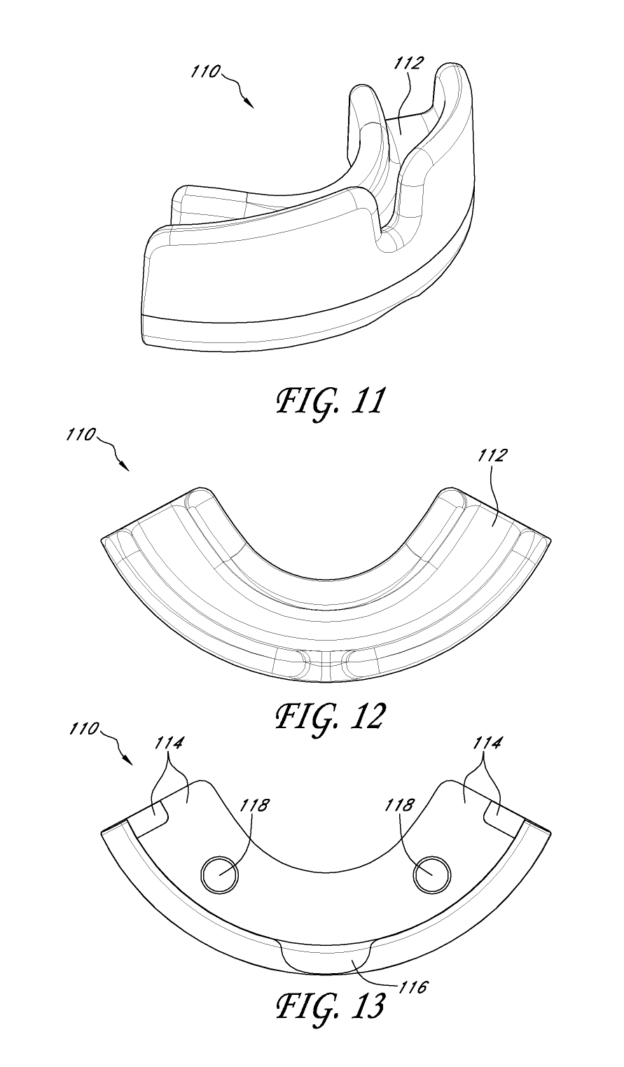

As can be seen in a top, back, and side view of the mouth securement member 110, illustrated in FIGS. 12, 15, and 16, respectively, the mouth securement member 110 comprises a receiving space 112. In the illustrated embodiment, the receiving space 112 is of a generally curved shape, which is approximately complementary to a patient's gums and/or teeth. Thus, the receiving space 112 may receive the patient's gums and/or teeth and maintain them in a close proximity to the mouth securement member 110, thereby reducing movement of the mouth securement member 110. In one embodiment, the mouth securement member 110 may comprise a moldable material, for example placed in the receiving space 112, which is configured to mold to the shape of the patient's gums and/or teeth. Alternatively, the mouth securement member 110 may already have been pre-molded to the shape of the patient's gums and/or teeth via, for example, a custom insert or the like.

In the illustrated embodiment, the mouth securement member 110 is configured to attach to the distribution member 60 in complement with the porous structures 94 and 96. As can be seen in the bottom view, back view, and side view of the mouth securement member 110, illustrated in FIGS. 13, 15, and 16, respectively, the bottom of the mouth securement member 110 is configured to form a plurality of receptacles 114. The receptacles 114 receive a portion of the porous structures 94 and 96 when the mouth securement member 110 and the porous structures are attached to the distribution manifold 60. The securement member 110 may also comprise additional receptacles, such as to accommodate the distribution manifold 60.

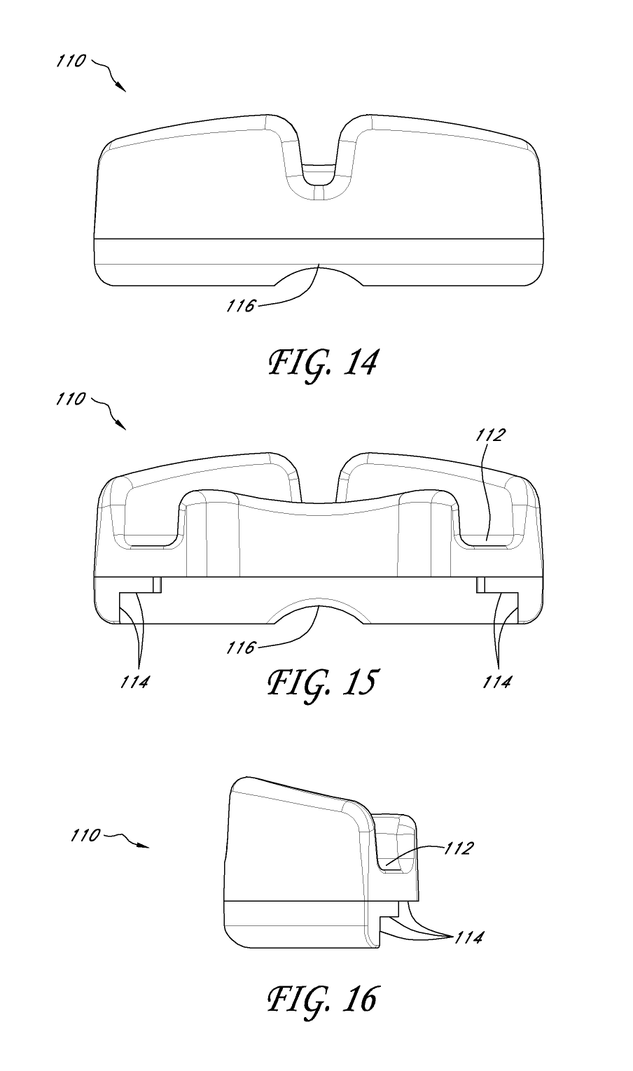

In the illustrated embodiment, the mouth securement member 110 is configured to attach to the top of distribution manifold 60. As can be seen in the bottom view of the mouth securement member 110, the mouth securement member 110 includes a plurality of recesses 118. The recesses 118 receive the protrusions 66 on the distribution manifold 60. As can be seen in the front and back views of the mouth securement member 110, illustrated in FIGS. 14 and 15, respectively, a depression 116 is formed in the mouth securement member 110. The depression 116 is configured to accommodate the connection member 56 or an endotracheal tube passing under the mouth securement member 110. In other embodiments, the mouth securement member 110 and connection member 56 may be configured such that the mouth securement member 110 attaches to the bottom of the connection member 56, or such that a mouth securement member can be connected to both the top and the bottom of the connection member 56. When a securement member is connected to both the top and the bottom of the connection member 56, both of a patient's upper and lower teeth and/or gums can be received.

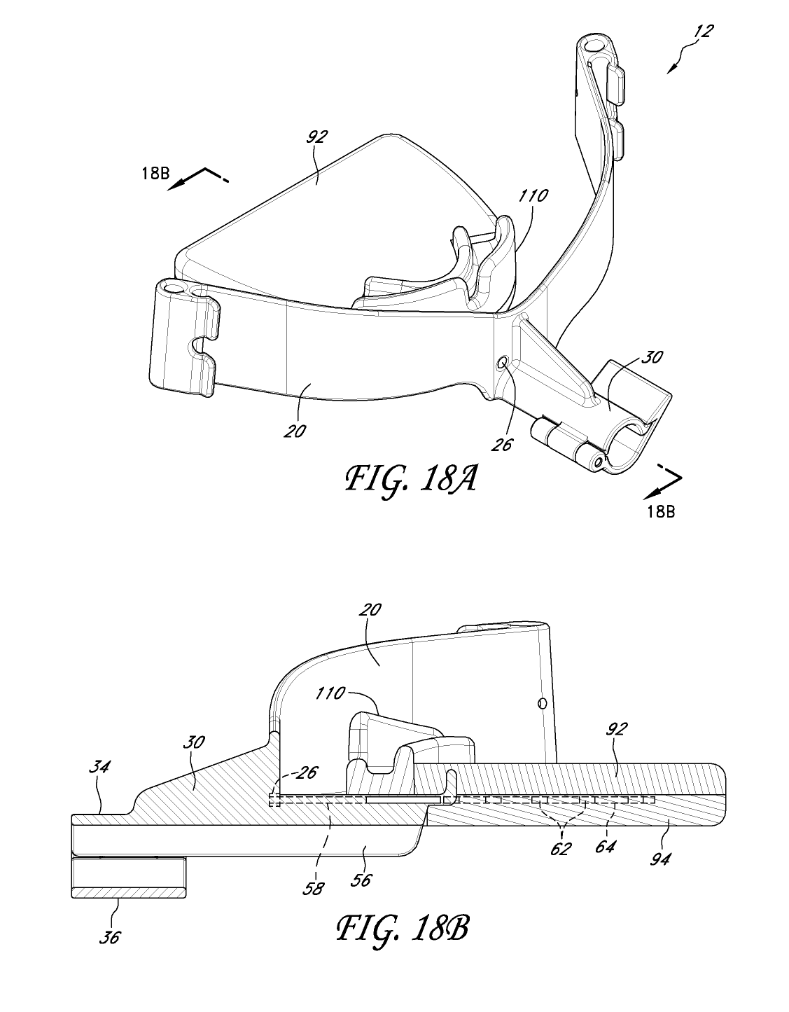

As can be seen in an exploded view of the securement system 10, illustrated in FIG. 17 without the flexible bands 190a and 190b, the securement system 10 may be assembled from the retainer 12, the porous structures 94 and 96, and the mouth securement member 110. The porous structure 94 and the mouth securement member 110 may be attached to the top of the distribution manifold 60, and the porous structure 96 may be attached to the bottom of the distribution manifold 60. When the attachments are complete, a structure for further distributing a liquid within the mouth of the patient will be assembled, as illustrated in a perspective view in FIG. 18a. Those skilled in the art will appreciate that a greater or fewer number of structures may be attached the retainer 12 than those illustrated. For example, the securement system 10 may comprise only one porous structure instead of two, the mouth securement element 110 may be omitted, or the securement system 10 may comprise three or more porous structures of varying shapes and/or sizes.

In one embodiment, when the distribution manifold 60 is assembled using the upper porous structure 92, the lower porous structure 94, and the mouth securement member 110, at least one of the porous structures 92 and 94 may be suspended over a surface of the distribution manifold 60 by one or more of the protrusions 68, thereby forming a cavity or gap between the surface of the distribution manifold 60 and the porous structure. In this way, a greater amount of liquid administered to the distribution manifold 60 can gather between the two porous structures 92 and 94, and liquid may freely pass through the apertures 62 to the porous structures 92 and 94.

In the illustrated embodiment where the distribution manifold 60 is configured with at least one lumen 64 therein, there is fluid communication between the aperture 26, the lumen portion 58 in the connection member 56, and the apertures 62. As can be seen in a cross-sectional view of the assembled securement system 10 without the flexible bands 190a and 190b illustrated in FIG. 18b, a liquid ejected into aperture 26 will thereafter flow through the lumens 64 and exit from upper and/or lower apertures 62 into the porous structure 92 and/or the porous structure 94. In addition, a portion of the lumen 64 may be disposed in the mouth securement member 110. Such an arrangement may facilitate liquid communication between the aperture 26 and the apertures 62.

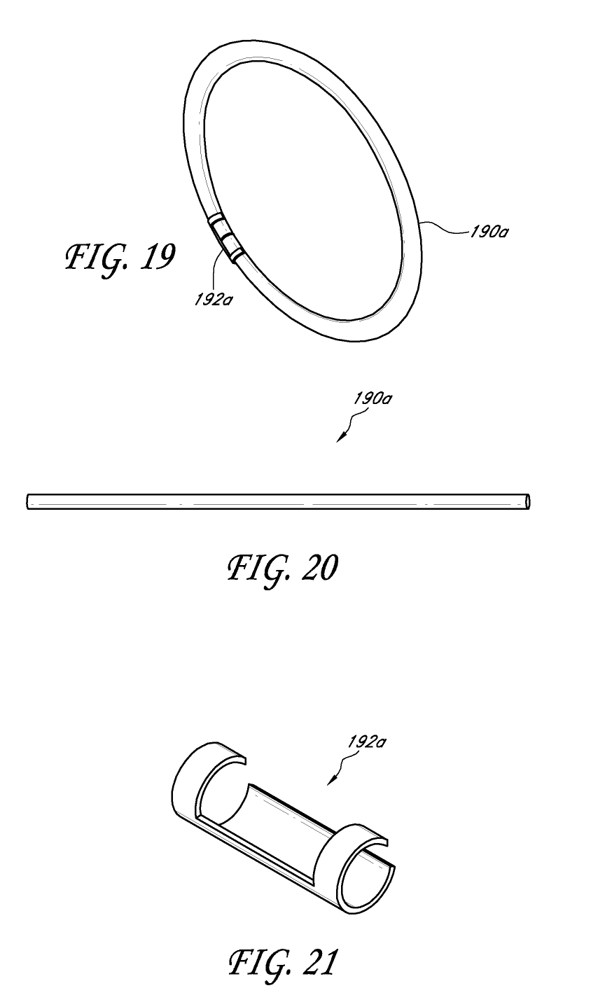

With reference now to FIG. 19, an embodiment of the flexible band 190a is illustrated. In combination with the flexible band 190b, which is illustrated in FIG. 1 and may be configured similar to the flexible band 190a, the flexible band 190a is configured to secure the support member 20, and thereby the securement system 10, relative to a patient's face. In order to secure the support member 20, the flexible bands 190a and 190b are connected to the support member 20 and then looped over the patient's ears, thereby encircling the patient's ears. Alternatively, the flexible bands 190a and 190b may be placed over the patient's ears and thereafter connected to the support member 20.

The flexible band 190a may comprise any filamentary element or material that is flexible and suitable for contact with a patient's skin, such as a compliant plastic tube. In some embodiments, the flexible band 190a may be a stretchable or deformable material, which may increase the ease of securing the flexible band 190a about the ear of the patient and increase the tension in the flexible band 190a once secured. For example, the flexible band 190a may comprise an elastic material, such as a rubber strap.

In some embodiments, the flexible band 190a may be a noncontiguous length of material, such as illustrated in FIG. 20, which may be configured as a loop using an adjustment member 192. The adjustment member 192 may not only be used to secure the length of material into a loop, as illustrated in FIG. 19, but in some embodiments may also or alternatively be used to adjust the circumference of the loop. In some embodiments, the adjustment member 192 may be attached to or integral with the support member 20.

The adjustment member 192, illustrated in FIG. 21, may be any clip, clasp, fastener, friction device, gripping object, or any other device that can be used to secure at least a portion of the flexible band 190a and/or adjust a circumference of a loop formed by the flexible band 190a. In the illustrated embodiment, the adjustment member 192 is configured as a clasp with flexible flanges. The adjustment member 192 may comprise a metal or plastic material, and may comprise a similar material as the support member 20.

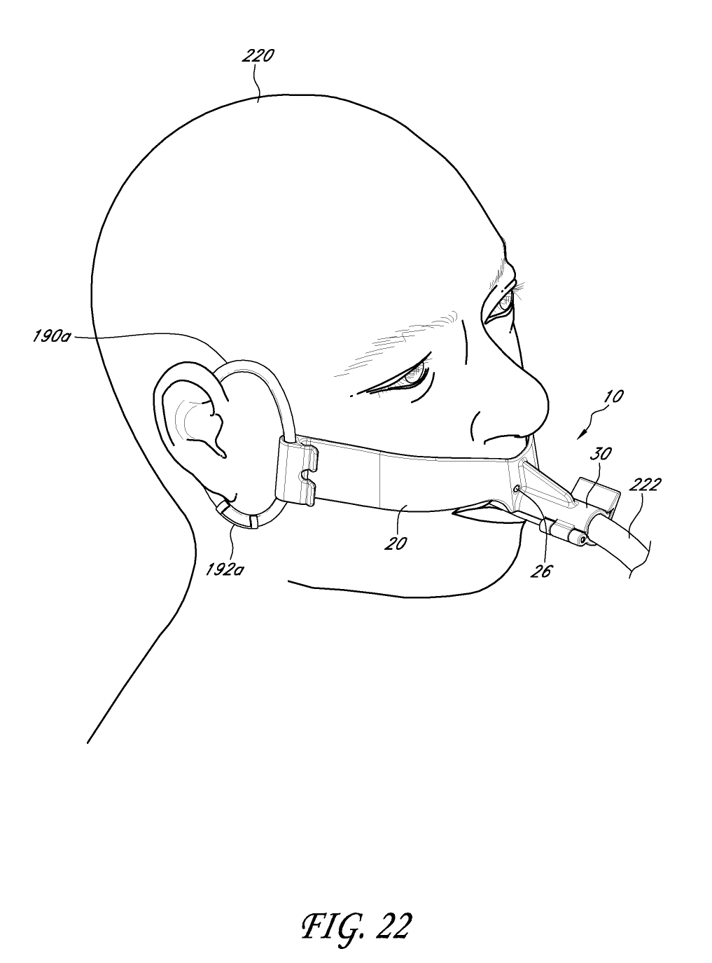

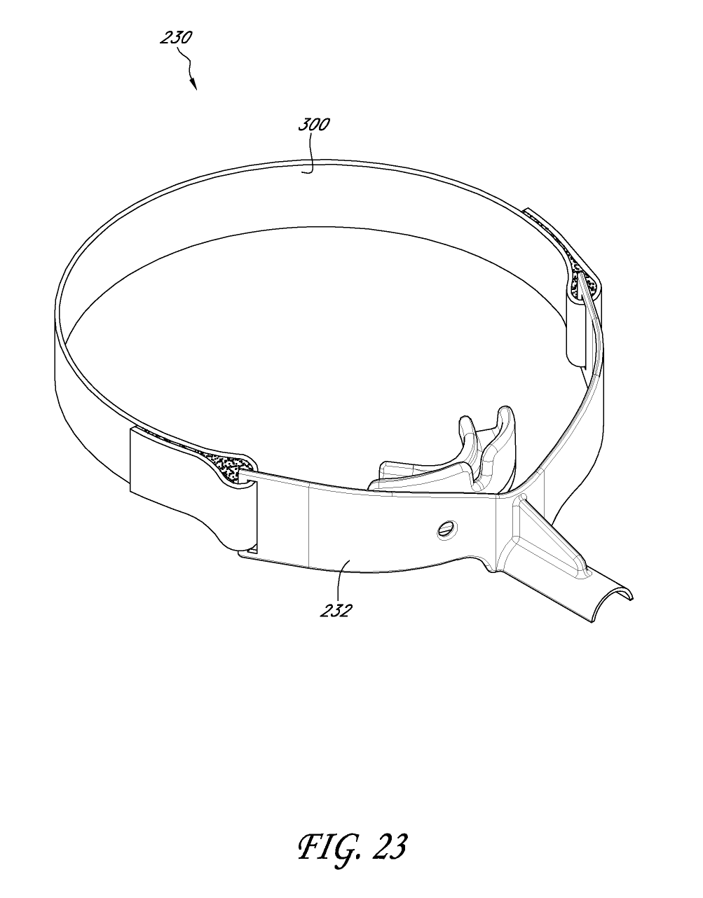

A perspective view of the securement system 10 secured to a patient 220 is illustrated in FIG. 22. The support member 20 rests against the face of the patient 220, while the aligning member 30 receives a portion of an endotracheal tube 222. The securement system 10 aligns the endotracheal tube 222 laterally and/or transversely with the mouth of the patient 220. The aligning member 30 preferably also secures the endo-treacheal tube longitudinally with respect to the securement system 10.

The securement system 10 is secured to the patient 220 by the flexible bands 190a and 190b, which encircle the ears of the patient 220. In one embodiment, loops formed by flexible bands attached to the securement system 10 may be placed or stretched around the ears of the patient 220 after the support member has been placed in contact with the face of the patient 220, or after the aligning member has received a portion of the endotracheal tube 222. In another embodiment, loops formed by flexible bands may be placed or stretched around the ears of the patient 220 and then connected to the securement system 10. In yet another embodiment, noncontiguous flexible bands may be formed into loops around the ears of the patient 220.

The medical practitioner may adjust the placement of the securement system 10 or the fit of the securement system 10 against the face of the patient 220 using the adjustment member 192. The adjustment member 192 allows the medical practitioner to adjust the tension exerted by the flexible band 190a on the support member 20, such that the support member 20 can fit securely against the face of the patient 220.

In the illustrated embodiment, the adjustment member 192 is illustrated as being placed at the bottom of the flexible band 190a, but in other embodiments the adjustment member 192 can be located anywhere along the length of the flexible band 190a length or can be integral with the support member 20. Although not illustrated in FIG. 22, a similarly configured adjustment member 192b may be connected to the flexible band 190b. Alternatively adjustment members may be omitted from the securement system 10. Those of skill in the art will appreciate that adjustment of the securement system 10 may be achieved without removing the endotracheal tube 222 from the retainer 12 and/or without removing the securement system 10 from the patient 200. Similarly, those of skill in the art will appreciate that the endotracheal tube 222 may be adjusted without removing the securement system 10 from the patient 220.

With reference now to FIG. 23, another embodiment of a securement system 230 includes a retainer 232 and a strap 300. The retainer 232 is similar to the retainer 12 illustrated in FIG. 18a except that the entrance aperture 266 into the lumen 64 of the retainer 232 illustrated in FIG. 23 is disposed in the mouth securement member 260 on a proximal side of the support member 240. The lumen 64 need not extend through the connection member 262. Additional differences between the structure of the retainer 12 illustrated in FIG. 18a and the retainer 232 illustrated in FIG. 23 are detailed below. The strap 300 or head contact member is configured to be placed around a portion of the patient's head.

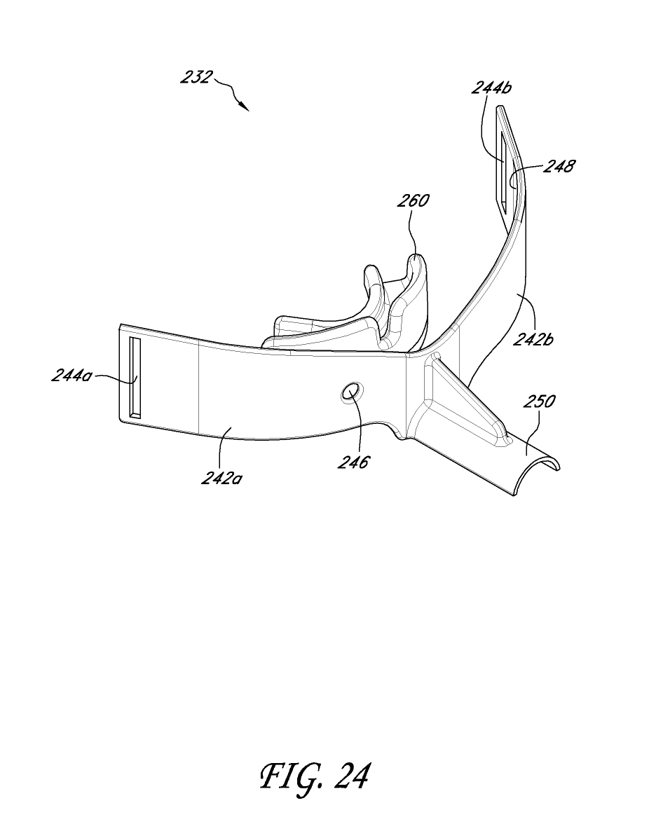

FIGS. 24-29 illustrate the retainer 232. As can be seen in a perspective view of the retainer 232, illustrated in FIG. 24, the retainer 232 includes a support member 240, an aligning member 250, and a mouth securement member 260. The support member 240 has a posterior facing surface 248 configured to inhibit movement of the retainer 232 into the patient's mouth. The aligning member 250 is configured to receive a portion of an endotracheal tube such that the tube is aligned with the patient's mouth. The mouth securement member 260 is configured for placement in the patient's mouth such that substantial movement of the securement system 230 is reduced.

The support member 240 is comprised of a first side 242a and a second side 242b. The shape and configuration of the support member 240 and of the sides 242a and 242b may be similar to the shape and configuration of the support member 20 and the sides 22a and 22b of the retainer 12, as described in reference to FIG. 2.

As can be seen in a back view of the support member 240, illustrated in FIG. 28, each side 242a and 242b of the support member 240 may comprise a pad 282a and 282b on the posterior surface 248 thereof. The pads 282a and 282b will directly contact the patient's face when the securement system 230 is secured to the patient. Such contact between the pads 282a and 282b may increase the comfort of the patient and/or may reduce excoriation or other adverse effects when the securement system 230 is secured to the patient. To realize increased comfort or decreased adverse effects, the pads 282a and 282b may be constructed of any material suitable for long-term contact with the patient's skin. In the illustrated embodiment, each side of the support member 240 comprises a single pad, but each side may comprise a greater number of pads. In addition, the securement system 10, illustrated in FIG. 1, may comprise pads similar to the pads 282a and 282b.

The proximal ends of the support member 240 are each define an opening 244a and 244b that accepts a portion of the strap 300 illustrated in FIG. 23. The size and shape of the openings 244a and 244b roughly correspond to a cross-section of the strap 300. Of course, the openings 244a and 244b can be larger than the cross-section of the strap 300. Matching a cross-section of the strap 300 to the size and shape of the openings 244a and 244b, however, minimizes longitudinal and/or transverse movement of the strap 300 when placed through the support member 240. In some embodiments, both openings 244a and 244b are a similar size and shape. In another embodiment, openings 244a and 244b may be sized or shaped differently.

The openings 244a and 244b are configured such that a length of the openings 244a and 244b is situated in a generally transverse direction. Thus, a strap placed through the openings 244a and 244b will extend from the support member 240 in the same general plane as the support member 240 and will not be substantially angled with respect to the support member 240. In addition, the openings 244a and 244b of the illustrated embodiment are approximately transversely centered on the support member 240, thereby allowing an even force to be applied to the support member 240 by the strap 300 when the strap 300 is placed through the openings 244a and 244b. In other embodiments, one or both of the openings 244a and 244b may be situated in a different direction or may be located in a different position.

According to various embodiments, the support member 240 may comprise additional openings that accept at least a portion of an additional strap or straps. Such additional straps may further reduce movement of the securement system 230 when the securement system 230 is secured to the patient. Such additional straps may be configured to be placed around a different portion of the patient's head than the strap 300. For example, the support member 240 may define additional openings, each one of which is transversely and/or longitudinally spaced from the openings 244a and 244b. The additional openings may also be angled relative to the openings 244a and 244b. The additional openings can be configured for retaining an additional strap that fits over the patient's head for added securement, such as over the crown or the top of a patient's head.

In one embodiment, the strap 300 and/or additional straps may be permanently attached to one end of the support member 240 and the other side may be configured to define one or more openings. In another embodiment, the strap 300 and/or additional straps may be permanently attached to both sides and may be made of a flexible material, such as elastic. In yet another embodiment, the securement system 230 may be configured to utilize the flexible bands 190a and 190b of securement system 10, illustrated in FIG. 1, in addition to or in place of the strap 300 and/or additional straps. Similarly, the securement system 10 may be configured to utilize the strap 300 and/or additional straps in addition to or in place of the flexible bands 190a and 190b.

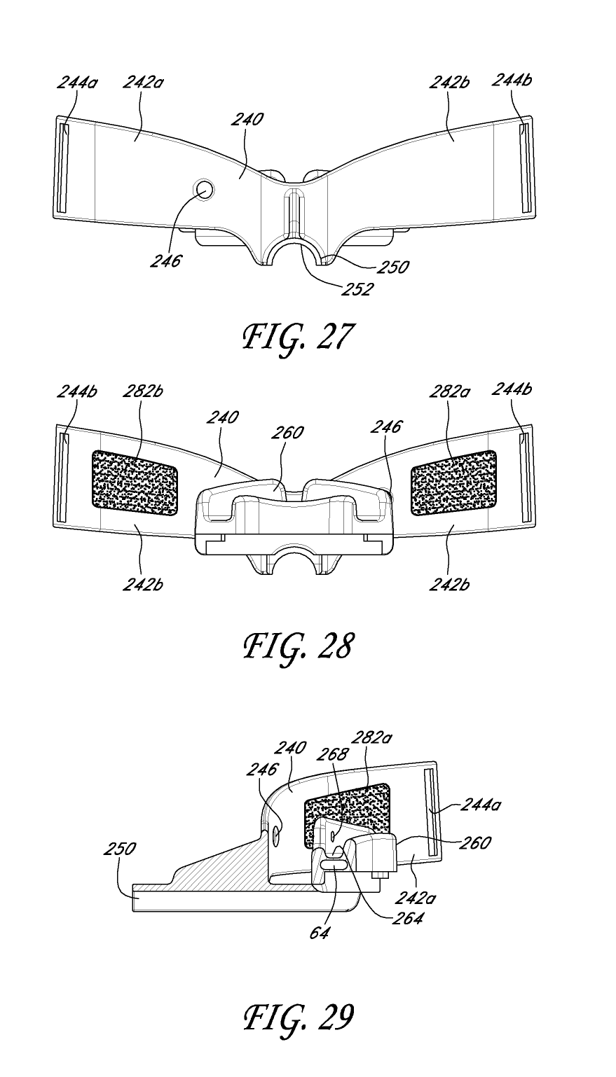

The support member 240 includes an aperture 246. The aperture 246 provides access to the patient's oral cavity from a distal side of the support member 240, thereby allowing infusion of a liquid into the patient's oral cavity. The aligning member 250 is attached to a distal/anterior side of the support member 240. As can be seen in a front view of the support member 240, illustrated in FIG. 27, the aligning member 250 has a channel 252 that is configured to receive a portion of an endotracheal tube.

As can be seen in FIG. 27, the aligning member 250 will not completely enclose a received endotracheal tube. Such configuration may reduce lateral movement of the tube and some transverse movement of the tube, while allowing a portion of the tube to be easily placed within the aligning member 250 and adjusted by a medical practitioner. In contrast to the embodiment of the aligning member 30 of the securement system 10 illustrated in FIG. 5, the illustrated aligning member 250 does not comprise a hinged member. In other embodiments, however, the aligning member 250 may comprise a hinged member, and a hinged member may be omitted from the aligning member 30.

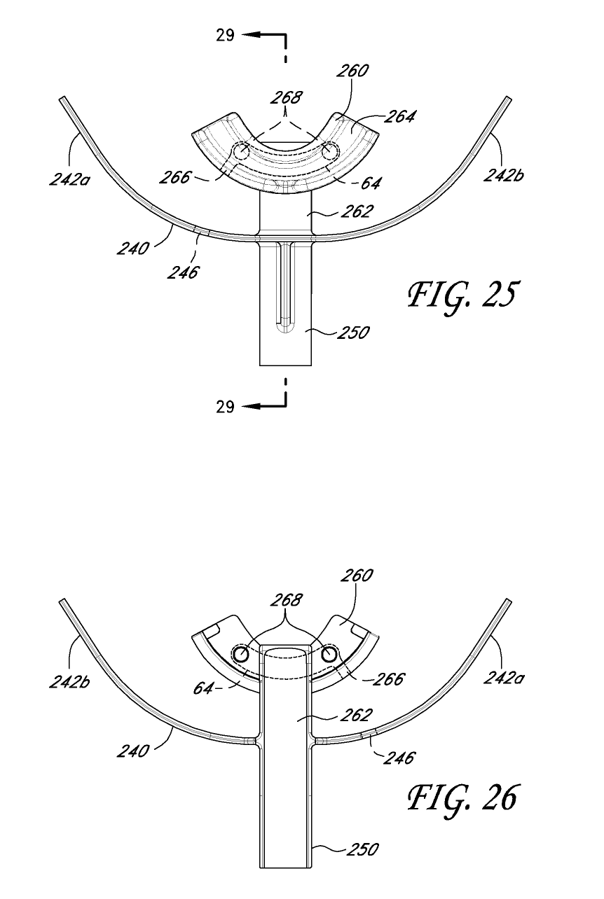

The mouth securement member 260 is attached to a proximal side of the support member 240, and may be attached by a connection member 262, as illustrated in a top view or bottom view of the support member 240 in FIG. 25 or FIG. 26, respectively. The connection member 262 may be configured similar to the connection member 56 of securement system 10, described in reference to FIG. 5. In the illustrated embodiment, the connection member 262 lacks a portion of the lumen 64 in contrast to the connection member 56.

As can be seen in a top and cross-sectional view of the mouth securement member 260, illustrated in FIGS. 25 and 29, respectively, the mouth securement member 260 comprises a receiving space 264. In the illustrated embodiment, the receiving space 264 is of a generally curved shape, which is approximately complementary to a patient's gums and/or teeth.

As can also be seen in the top and cross-sectional views of the mouth securement member 260, illustrated in FIGS. 25 and 29, respectively, the mouth securement member 260 is configured to define an aperture 266 in the front thereof. The aperture 266 is positioned so that a medical practitioner can place a portion of a syringe, or other instrument used to dispense liquid, through the aperture 246 in the support member 240 and flush a liquid through the aperture 266. Thus, the apertures 246 and 266 are approximately aligned.

As can be seen in the top and bottom views of the mouth securement member 260, illustrated in FIGS. 25 and 26, the mouth securement member 260 may additionally be configured to define one or more apertures 268 in the bottom thereof. A liquid, such as an antimicrobial agent, flushed into the aperture 266, will be infused into the patient's oral cavity through the one or more apertures 268. Alternatively, the aperture 268 may be in lumen connection with the aperture 246, such as described in reference to the apertures 62 being in lumen connection with the aperture 26 of the securement system 10. Conversely, the distribution manifold 60 and/or the mouth securement member 110 of the securement system 10 may have an aperture therein which is aligned with the aperture 26 similar to how the aperture 266 and the aperture 246 are aligned, thereby allowing a medical practitioner to place a portion of a syringe or other instrument through the aperture 26 and flush a liquid through the aligned aperture.

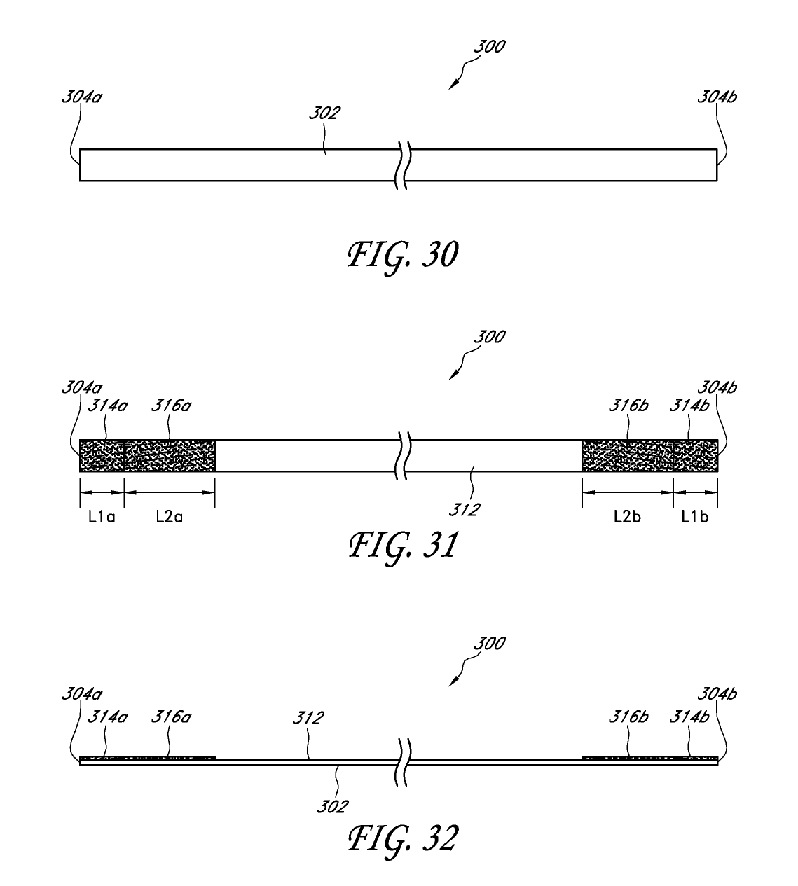

With reference now to FIGS. 30-32, an embodiment of the strap 300 is illustrated. As illustrated in FIG. 30, the strap is configured such that at least the ends 304a and 304b of the strap 300 may be passed through the openings 244a and 244b of the support member 240, illustrated in FIG. 24. In the illustrated embodiment, the strap 300 is further configured to be placed around the back of a patient's head and/or neck. Thus, a length of the strap 300 allows the strap 300 to pass through the opening 244a of the support member 240, travel around the back of a patient's head and/or neck, and pass through the opening 244b of the support member 240. In addition, an upper surface 302 of the strap 300 is configured to comfortably be placed in contact with a patient's hair and/or skin.

The shape and construction of the strap 300 may otherwise be varied. Any number of shapes or designs of the strap 300 are possible and within the scope of this description. For example, although the strap 300 is illustrated as being substantially uniform in width, the strap 300 may be shaped so as to be wider at its middle to provide a greater contact area with the head and/or neck of the patient. Although the strap 300 is illustrated as having squared ends 304a and 304b, which may facilitate a secure connection when the strap 300 is attached to the support member 240, other embodiments include a strap with ends that are not squared with a single squared end. One such embodiment is a strap with rounded ends, which may ease placing the strap 300 through the openings 244a and 244b. Although the strap 300 is illustrated as a single piece of material, the strap 300 may also comprise several pieces of material attached together.

Viewed from the bottom, as illustrated in FIG. 31, the strap 300 is comprised of a lower surface 312, hook portions 314a and 314b, and loop portions 316a and 316b. In one embodiment, the hook portions 314a and 314b and the loop portions 316a and 316b are attached onto the lower surface 312, as illustrated in FIG. 32. In another embodiment, the lower surface 312 may only extend to the beginning of the hook portions 314a and 314b or the loop portions 316a and 316b, and the remaining portions may be attached to each other laterally instead of placed on the lower surface 312. Thus, the strap 300 may be comprised of multiple sections or portions attached together. The lower surface 312 may be integral to the upper surface 302, illustrated in FIG. 30, or the lower surface 312 may be separate from and stacked on or laminated to the upper surface 302. In one embodiment, at least a part of the lower surface 312 or similarly the upper surface 302—comprises a stretchable or deformable material, which may increase the ease of securing the strap 300 about the head of the patient and increase the tension in the strap 300 once secured. For example, the lower surface 312 may comprise an elastic material.

The hook portion 314a and the loop portion 316a are sequentially disposed on the end 304a of the strap 300, and the hook portion 314b and the loop portion 316b are sequentially disposed on the end 304b of the strap 300. When the end 304a is placed through the opening 244a or 244b, for example, the end 304a can be folded back toward the rest of the strap 300 and the hook portion 314a can mate with the loop portion 316a, thereby attaching the strap 300 to the support member 240. The hook portion 314b can similarly mate with the loop portion 316b when the end 304b is placed through the opening 244a or 244b.

Either the hook portion 314a or the loop portion 316a may be located next to the end 304a. FIG. 31 illustrates the hook portion 314a as being located next to the end 304a, but the placement of the hook portion 314a and the loop portion 316a could be reversed, with the loop portion 316a being located next to the end 304a. The hook portion 314a and the loop portion 316a may directly abut each other or there may be a distance between the portions.

The hook portion 314a and/or the loop portion 316a can span the entire width of the strap 300. In another embodiment, the hook portion 314a and/or the loop portion 316a may be configured as various shapes or may only partially cover the width of the strap 40.

The hook portions 314a and the loop portion 316a have lengths L1a and L2a, respectively, such that when the strap 300 is attached to the support member 240 and placed around the patient's head, at least a portion of the hook portion 314a can be passed through the opening 244a or 244b and folded back to contact at least a portion of the loop portion 316a. The hook portion 314b and the loop portion 316b may be configured similar to the hook portion 314a and the loop portion 316a with respect to each other and with respect to the end 304b.

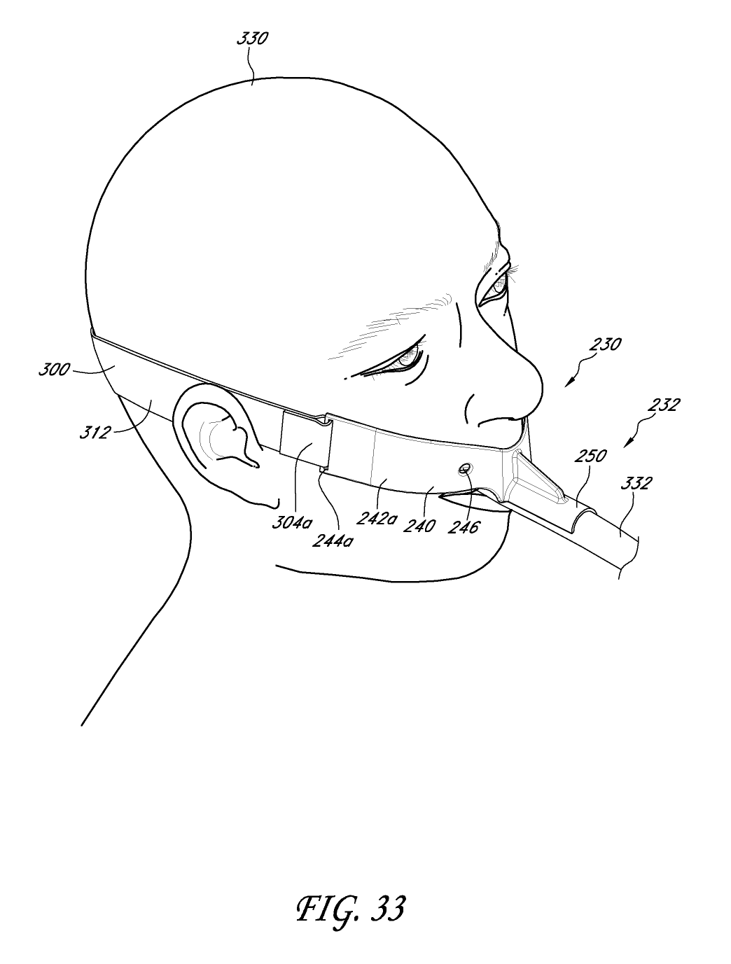

A perspective view of the securement system 230 secured to a patient 330 is illustrated in FIG. 33. The support member 240 rests against the face of the patient 330, while the aligning member 250 receives a portion of an endotracheal tube 332 and aligns the endotracheal tube 332 with the mouth of the patient 330.

The securement system 230 is secured to the patient 220 by the strap 300. A medical practitioner may place the end 304a through the opening 244a by, for example, positioning the lower surface of the strap 300 to face away from the support member 240. The medical practitioner can fold the end 304a back towards the strap 300, causing the hook portion 314a to mate with the loop portion 316a. The medical practitioner can draw the strap 300 around the back of the head or neck of the patient 330 and then place the end 304b through the opening 244b, for example. The end 304b can then be folded back towards the strap 300, causing the hook portion 314b to mate with the loop portion 316b, thereby attaching the strap 300 to the support member 240 and securing the securement system 230 to the patient 330.

In another embodiment, at least one end of the strap 300 may be permanently attached to the support member 240. In such an embodiment, the medical practitioner can draw the other end, i.e. the free end, of the strap 300 around the back of the head or neck of the patient 330 and then place the free end through an opening in the support member 240. Subsequently, the free end can then be folded back towards the strap 300, causing a hook portion near the free end to mate with a loop portion near the free end. If both ends of the strap 300 are permanently attached to the support member 240, the medical practitioner may start by placing the securement system 230 over the top of the head of the patient 330. Then, the medical practitioner may pull the support member 240 down to the proper position on the face of the patient 330, while simultaneously pulling or stretching the strap 300 around the head and/or neck of the patient 330.

When both ends of the strap 300 are not permanently attached to the support member 240, the medical practitioner may adjust the placement of the securement system 230 or the fit of the securement system 230 against the face of the patient 330 using the hook portions 314a and 314b and the loop portions 316a and 316b. The hook portions 314a and 314b and the loop portions 316a and 316b permit adjustment to the length of the strap 300, such that the pads 282a and 282b can be properly tensioned against the face of the patient 330. Those of skill in the art will appreciate that adjustment of the securement system 230 may be achieved without removing the endotracheal tube 332 from the aligning member 250 and/or without removing the securement system 230 from the patient 330. Similarly, those of skill in the art will appreciate that the endotracheal tube 332 may be adjusted without removing the securement system 230 from the patient 330.

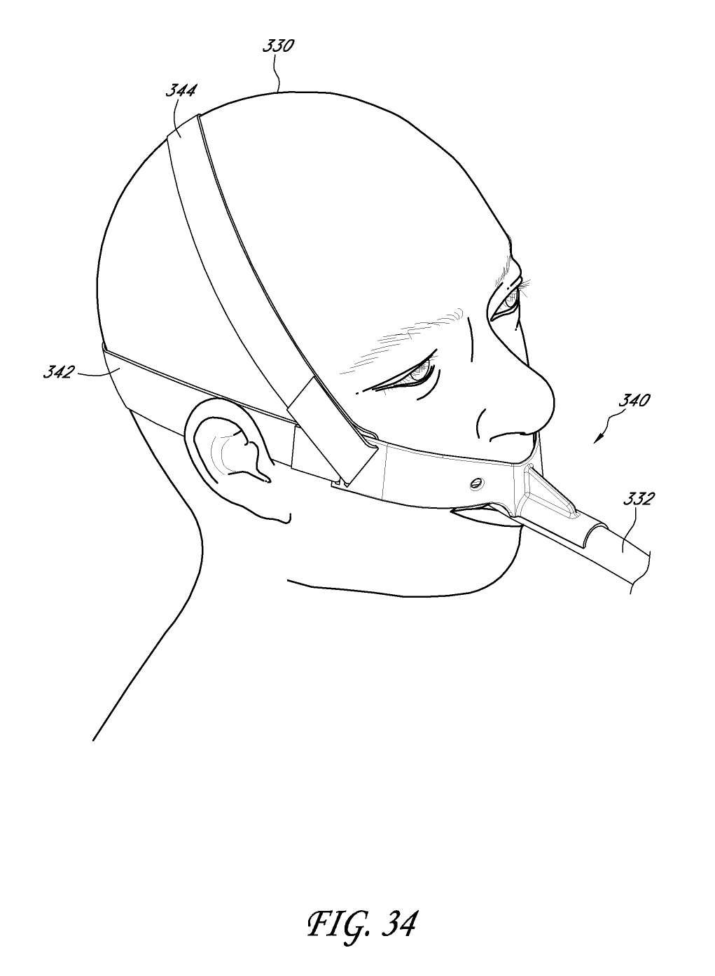

A perspective view of another securement system 340 secured to the patient 330 is illustrated in FIG. 34. The securement system 340 differs from the securement system 230 in that the securement system 340 comprises straps 342 and 344, in contrast to the single strap 300 in the illustrated embodiment of the securement system 230. Both of the straps 342 and 344 are configured to be placed around the head and/or neck of the patient 330 and may be configured similar to the strap 300. The strap 342 may be placed around head and/or neck of the patient 330 in a position similar to that of strap 300 or may be placed around the head and/or neck of the patient 330 in a position different from that of strap 300. The strap 344 may be placed around a portion of the head and/or neck of the patient 330 different from the position of strap 342, such as around the crown or the top of the head of the patient 330. The addition of the strap 344 may further decrease the likelihood of substantial movement of the securement system 340 when secured to the patient 330.

The various embodiments of the securement systems described above in accordance with the present invention thus provide a means to secure an endotracheal tube or other medical line to a patient and to infuse a liquid into the oral cavity of the patient when the medical line is secured to the patient. The endotracheal tube can be adjusted without removing the entire securement assembly, and without the need for use of additional tape to re-secure the endotracheal tube once it is properly repositioned.

Those of skill in the art will appreciate that embodiments of the securement system can be used not only to infuse a liquid into the oral cavity of the patient, but also to remove liquid from the oral cavity. In some embodiments, a section device is attached to the securement system such that the suction device is in flow communication with the lumen. The suction device can then be used to withdraw liquid from the oral cavity. The suction device may be any device configured to attach to the securement system and configured to draw liquid, such as a vacuum pump.

Of course, it is to be understood that not necessarily all objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Furthermore, the skilled artisan will recognize the interchangeability of various features from different embodiments. In addition to the variations described herein, other known equivalents for each feature can be mixed and matched by one of ordinary skill in this art to construct securement systems and techniques in accordance with principles of the present invention.

Although this invention has been disclosed in the context of certain embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above.

What is claimed is:

1. A retainer for an endotracheal tube, the retainer comprising:
a posterior facing surface configured to inhibit movement of the retainer into a person's mouth;
a channel configured to receive a portion of the endotracheal tube so as to inhibit movement of the endotracheal tube relative to the retainer;
a distribution manifold, at least a portion of the distribution manifold being configured to be disposed in the person's mouth; and
a lumen, at least a portion of the lumen being disposed in the retainer so that a liquid passing through the lumen enters the person's mouth.

2. The retainer of claim 1, wherein at least a portion of the lumen extends through the distribution manifold.

3. The retainer of claim 1 further comprising an exit aperture, the exit aperture being disposed in the distribution manifold and in flow communication with the lumen.

4. The retainer of claim 3 further comprising a porous structure covering the exit aperture, wherein the porous structure is configured such that liquid can be communicated from the exit aperture into the person's mouth.

5. The retainer of claim 1, wherein at least a portion of the lumen forms a closed passageway into the person's mouth.

6. The retainer of claim 1 further comprising an opening in a wall of at least a portion of the lumen.

7. The retainer of claim 1, wherein a cross-section of the lumen varies along a length of the lumen.

8. The retainer of claim 1, wherein the lumen transitions between multiple shapes along its length.

9. The retainer of claim 1, wherein the lumen comprises one or more manifolds or pockets.

10. The retainer of claim 1, wherein the lumen has a tapering inner surface and a radius that narrows in a direction towards the person's mouth.

11. The retainer of claim 1, wherein the retainer comprises multiple lumens.

12. The retainer of claim 11, wherein a plurality of the multiple lumens are interconnected.

13. The retainer of claim 12, wherein the interconnected plurality of lumens form one or more branching sites.

14. The retainer of claim 1 further comprising a connection member.

15. The retainer of claim 14, wherein at least a portion of the lumen extends through at least a portion of the connection member.

16. The retainer of claim 1 further comprising a plurality of pads residing on the posterior facing surface.

17. The retainer of claim 1, wherein the channel comprises a stationary member and a hinged member, and wherein the hinged member is configured to engage with the stationary member.

18. The retainer of claim 1, wherein the channel circumscribes at least a portion of the endotracheal tube.

19. The retainer of claim 1 further comprising a flexible band.

20. An endotracheal tube securement system comprising:
a head contact member configured to be secured to a patient; and
a retainer securable to the head contact member and comprising:
a support member having a posterior facing surface configured to inhibit movement of the retainer relative to a patient's mouth;
an aligning member configured to receive a portion of the endotracheal tube so as to inhibit movement of the endotracheal tube in at least one direction relative to the support member; and
a lumen disposed so that a liquid passing through the lumen enters the person's mouth.

21. The system of claim 20 further comprising a suction device in flow communication with the lumen.

22. The system of claim 21, wherein the suction device is a vacuum pump.

23. A retainer for securing an endotracheal tube to a patient, the retainer comprising:
an alignment member configured to inhibit movement of the endotracheal tube relative to the retainer;
a first opening configured to be disposed within a patient's mouth; and
a second opening configured to be accessible from outside the patient's mouth and at least partially in flow communication with the first opening.

24. The retainer of claim 23, further comprising a lumen in flow communication with the first and second openings.

25. The retainer of claim 24, wherein at least a portion of the lumen is configured to pass between an upper jaw and a lower jaw of the patient when the retainer is secured to the patient.
